(19) United States
(12) Patent Application Publication
(10) Pub. No.: US 2025/0352155 A1
(43) Pub. Date: Nov. 20, 2025

(54) ARM SUPPORT FOR MEDICAL IMAGING AND TREATMENT

(71) Applicant: LEO CANCER CARE, INC., Middleton, WI (US)

(72) Inventors: Stephen Towe, East Sussex (GB); Ross Tierney, Middleton, WI (US); Niek Schreuder, Middleton, WI (US); Bethany O'Hanlon-Clayton, Middleton, WI (US)

(21) Appl. No.: 19/122,314

(22) PCT Filed: Oct. 20, 2023
(86) PCT No.: PCT/US2023/035582

§ 371 Date: Apr. 18, 2025

Related U.S. Application Data
(60) Provisional application No. 63/418,128, filed on Oct. 21, 2022.

(51) Int. Cl. A61B 6/04; A61G 7/075

(52) U.S. Cl. CPC A61B 6/0407; A61G 7/075

(57) ABSTRACT

A bracket assembly for an arm support, the bracket assembly comprising: a first panel; a second panel coupled to the first panel at a pivot, a support channel configured to receive at least a portion of the arm support; and a clamping channel at least partially defined between the first panel and the second panel. The bracket assembly is movable between a first configuration where the first panel is movable relative to the second panel about the pivot and a second configuration where the first panel is fixed relative to the second panel.

### RELATED APPLICATIONS

This application claims priority to and the benefit of U.S. Provisional Patent Application No. 63/418,128 filed on Oct. 21, 2022, which is incorporated herein by reference in its entirety for all purposes.

### FIELD

Provided herein is technology relating to radiological imaging and treatment and particularly, but not exclusively, to an arm support configured to support the arms of a patient positioned in an upright position during medical imaging and/or during medical treatment and to related methods, kits, and systems.

### BACKGROUND

Radiation sources have many uses in medicine, including medical imaging and radiation therapy. Generally, radiation sources are configured to move in relation to a stationary patient, e.g., to expose a specific part or region of the patient to radiation produced by the source. Further, while radiation therapy and the associated diagnostic and planning imaging are conventionally performed with patients in prone or supine horizontal positions, some patients benefit from therapy while in an unconventional position, such as an upright position. Some patient supports used to position and support a patient include arm supports to position, support, and/or immobilize the patient's arms during diagnosis, imaging, and/or treatment. For example, arm supports may aid in supporting the patient's body in general or may place the patient's arms in a position that minimizes attenuation of the beam by the arms, minimizes exposure of body regions that are not being imaged or treated, or that maximizes imaging and treatment quality for body regions that are being imaged or treated. However, limitations of patient supports for supporting patients in upright positions have hindered adoption and use of beneficial upright radiotherapy methods. For example, upright positioning systems have been demonstrated to have reduced positional reproducibility relative to horizontal patient positioning. See, e.g., Rahim et al. (2020) Frontiers in Oncology 10, article 213, incorporated herein by reference. In particular, some arm supports for imaging and/or treating a patient in an upright position are complicated (see, e.g., Int'l Pat. App. Pub. No. WO2018120604) and thus add time needed for setting up the patient support, for positioning a patient, and for patient egress from the patient support, which all combine to lengthen the total imaging or treatment time and thus decrease overall patient throughput and treatment. Accordingly, improved patient arm supports are needed.

### SUMMARY

Provided herein is technology relating to radiological imaging and treatment and particularly, but not exclusively, to an arm support configured to support the arms of a patient positioned in an upright position during medical imaging and/or during medical treatment and to related methods, kits, and systems. Some technologies for supporting a patient a patient in an upright, stable position are described in U.S. Pat. App. Pub. No. 20200268327 and in U.S. Pat. App. Ser. No. 63/237,513, each of which is incorporated herein by reference. In some embodiments, the technology described herein supplements and/or modifies a patient positioning apparatus and/or a patient support, e.g., as described in U.S. Pat. App. Pub. No. 20200268327 or in U.S. Pat. App. Ser. No. 63/237,513, each of which is incorporated herein by reference. In some embodiments, a patient positioning system stabilizes and supports a patent in an upright (e.g., standing, sitting, kneeling, perched) position. Imaging and/or treating patients in an upright position provides the benefits of increasing patient comfort. Further, diagnosis and/or treatment of patients in an upright position provides advantages over conventional diagnosis and/or treatment of patients in a horizontal position for many indications (e.g., lung cancer, breast cancer). While imaging and/or treating an upright patient provides diagnostic and therapeutic advantages, medical imaging and treatment needs improved patient positioning systems for stabilizing and supporting patients in a suitable upright position for delivering therapeutic radiation doses to target areas and for planning treatment using medical imaging. The technology provided herein relates to improved arm supports for a patient positioning system that supports a patient in an upright position.

In one aspect, the disclosure provides a bracket assembly for an arm support, the bracket assembly comprising: a first panel; a second panel coupled to the first panel at a pivot, a support channel configured to receive at least a portion of the arm support; and a clamping channel at least partially defined between the first panel and the second panel. The bracket assembly is movable between a first configuration where the first panel is movable relative to the second panel about the pivot and a second configuration where the first panel is fixed relative to the second panel.

In some embodiments, the bracket assembly further includes a fastener to selectively place the bracket assembly in the second configuration.

In some embodiments, the first panel includes a first protrusion and a second protrusion and the support channel is at least partially defined by the first protrusion and the second protrusion.

In some embodiments, the first protrusion has a first surface configured to engage the arm support, and the second protrusion has a second surface configured to engage the arm support. The first surface is spaced from and facing towards the second surface.

In some embodiments, the bracket assembly further includes a detent formed on the first surface of the first protrusion.

In some embodiments, the first panel includes a first side portion extending between the first protrusion and the second protrusion, and a second side portion extending between the first protrusion and the second protrusion. The first side portion is spaced from and parallel to the second side portion.

In some embodiments, the support channel is at least partially defined between the first side portion, the second side portion, the first protrusion, and the second protrusion.

In some embodiments, the bracket assembly further includes a detent positioned within the support channel.

In some embodiments, in the first configuration the first panel is pivoted further away from the second panel than when in the second configuration.

In some embodiments, the support channel defines a support axis and the pivot defines a pivot axis and wherein the support axis is perpendicular to the pivot axis.

In some embodiments, the clamping channel is at least partially defined by a planar surface on the first panel and an arcuate surface of the second panel. The planar surface is positioned facing towards the arcuate surface.

In some embodiments, the bracket assembly further includes a detent formed in the clamping channel.

In some embodiments, the detent is at least partially formed by the first panel and the second panel when the bracket assembly is in the second configuration.

In one aspect, the disclosure provides an arm support assembly comprising: a first bracket assembly; a second bracket assembly; and an arm support. The arm support includes a rod having a first linear portion, a second linear portion, and a connecting portion positioned between the first linear portion and the second linear portion. The first linear portion is at least partially received in the first bracket assembly and the second linear portion is at least partially received in the second bracket assembly.

In some embodiments, the arm support includes a tray coupled to the rod.

In some embodiments, the tray is coupled to the connecting portion.

In some embodiments, the tray is a first tray and the arm support includes a second tray coupled to the connecting portion, wherein the first tray is spaced apart from the second tray.

In some embodiments, a handle grip extends between the first tray and the second tray.

In some embodiments, the handle grip is movable with respect to the first tray and the second tray.

In some embodiments, a distance between the arm support and the first bracket assembly is adjustable.

In some embodiments, the distance is between the first bracket assembly and the connecting portion.

In some embodiments, the first bracket assembly and the rod form a first detent arrangement, and the second bracket assembly and the rod form a second detent arrangement.

In some embodiments, the first detent arrangement includes a protrusion on the first bracket assembly and an aperture on the rod.

In some embodiments, the connecting portion is arcuate.

In some embodiments, the arm support is movable with respect to the first bracket assembly and the second bracket assembly between an in-use position and a storage position.

In one aspect, the disclosure provides an adjustable support assembly configured to support a patient. The adjustable support assembly comprising: a backrest; a first bracket assembly adjustably coupled to the backrest; a second bracket assembly adjustably coupled to the backrest; and an arm support adjustably coupled to the first bracket assembly and adjustably coupled to the second bracket assembly. A first adjustable dimension for the patient is defined between the first bracket assembly and the backrest, and a second adjustable dimension for the patient is defined between the arm support and the backrest.

In some embodiments, the first adjustable dimension and the second adjustable dimension are the only adjustable dimensions for locating the arm support with respect to the patient.

In some embodiments, the arm support includes a rod with a first portion and a second portion. The first portion is at least partially received within the first bracket assembly and the second portion is at least partially received within the second bracket assembly.

In some embodiments, the first bracket assembly is coupled to a first edge of the backrest and the second bracket assembly is coupled to a second edge of the backrest, the second edge opposite the first edge.

In some embodiments, the backrest is vertical.

In one aspect, the disclosure provides a method of adjusting a patient support to accommodate a patient, the method comprising: adjusting a first height of a first bracket assembly with respect to a backrest; adjusting a second height of a second bracket assembly with respect to the backrest; inserting an arm support at least partially in the first bracket assembly and at least partially in the second bracket assembly; and adjusting a spacing of the arm support with respect to the backrest.

In some embodiments, adjusting the spacing of the arm support with respect to the backrest is perpendicular to adjusting the first height of the first bracket assembly with respect to the backrest.

In some embodiments, the method further includes moving the arm support between an in-use position and a storage position.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A illustrates a patient positioning system supporting a patient in a perched position for treatment in, for example, the pelvis or lower abdomen.

FIG. 1B illustrates a patient positioning system supporting a patient in a seated position for treatment in, for example, the thorax or upper abdomen.

FIG. 1C illustrates a patient positioning system supporting a patient for treatment in, for example, the head or neck.

FIG. 2 is a perspective view of a vertical backrest with an arm support assembly. FIG. 3 is a perspective view of FIG. 2, with portions of the arm support assembly removed.

FIG. 4 is a perspective view of an arm support with a rod and a tray.

FIG. 5 is a partial perspective view of a bracket assembly in an open configuration.

FIG. 6 is a partial perspective view of the bracket assembly of FIG. 5 in a closed configuration.

FIG. 7 is a perspective cross-sectional view of the arm support assembly.

FIG. 8 is a side view of the arm support assembly illustrating the two adjustable dimensions of the arm support with respect to the backrest.

FIG. 9 is a perspective view of a front panel of a bracket assembly.

FIG. 10 is a partial perspective view of the front panel of FIG. 9.

FIG. 11 is another perspective view of the front panel of FIG. 9.

FIG. 12 is a perspective view of the rear panel of the bracket assembly.

FIG. 13 is a perspective view of an arm support assembly in a storage position, whereby the patient is free to enter or leave the upright backrest.

FIG. 14 is a perspective view of the arm support assembly of FIG. 13 in an in-use position, whereby a patient supports their arms on the arm support assembly during, for example, stationary beam therapy.

FIG. 15 is a perspective view of a vertical backrest with an arm support assembly including a vertical handle grip.

FIG. 16 is a perspective view of the arm support assembly of FIG. 15 in an in-use position, whereby a patient supports their arms on the arm support assembly during, for example, stationary beam therapy.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to radiological imaging and treatment and particularly, but not exclusively, to an arm support configured to support the arms of a patient positioned in an upright position during medical imaging and/or during medical treatment and to related methods, kits, and systems.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention is readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. is used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre determined threshold; conversely, when an entity is said to be “absent”, it means the level or amount of this entity is below a pre-determined threshold. The pre determined threshold is the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language is a compiled or interpreted language, and combined with hardware implementations.

As used herein, the term “computed tomography” is abbreviated “CT” and refers both to tomographic and non-tomographic radiography. For instance, the term “CT” refers to numerous forms of CT, including but not limited to X-ray CT, positron emission tomography (PET), single photon emission computed tomography (SPECT), and photon counting computed tomography. Generally, computed tomography (CT) comprises use of an X-ray source and a detector that rotates around a patient and subsequent reconstruction of images into different planes. In embodiments of CT (e.g., devices, apparatuses, and methods provided for CT) described herein, the X-ray source is a static source and the patient is rotated with respect to the static source. Currents for X-rays used in CT describe the current flow from a cathode to an anode and are typically measured in milliamperes (mA).

As used herein, the term “structured to [verb]” means that the identified element or assembly has a structure that is shaped, sized, disposed, coupled, and/or configured to perform the identified verb. For example, a member that is “structured to move” is movably coupled to another element and includes elements that cause the member to move or the member is otherwise configured to move in response to other elements or assemblies. As such, as used herein, “structured to [verb]” recites structure and not function. Further, as used herein, “structured to [verb]” means that the identified element or assembly is intended to, and is designed to, perform the identified verb.

As used herein, the term “associated” means that the elements are part of the same assembly and/or operate together or act upon/with each other in some manner. For example, an automobile has four tires and four hub caps. While all the elements are coupled as part of the automobile, it is understood that each hubcap is “associated” with a specific tire.

As used herein, the term “coupled” refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components. As used herein, “directly coupled” means that two elements are directly in contact with each other. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the term “removably coupled” or “temporarily coupled” means that one component is coupled with another component in an essentially temporary manner. That is, the two components are coupled in such a way that the joining or separation of the components is easy and does not damage the components. Accordingly, “removably coupled” components is readily uncoupled and recoupled without damage to the components.

As used herein, the term “operatively coupled” means that a number of elements or assemblies, each of which is movable between a first position and a second position, or a first configuration and a second configuration, are coupled so that as the first element moves from one position/configuration to the other, the second element moves between positions/configurations as well. It is noted that a first element is “operatively coupled” to another without the opposite being true.

As used herein, the term “rotatably coupled” refers to two or more components that are coupled in a manner such that at least one of the components is rotatable with respect to the other.

As used herein, the term “translatably coupled” refers to two or more components that are coupled in a manner such that at least one of the components is translatable with respect to the other.

As used herein, the term “temporarily disposed” means that a first element or assembly is resting on a second element or assembly in a manner that allows the first element/assembly to be moved without having to decouple or otherwise manipulate the first element. For example, a book simply resting on a table, e.g., the book is not glued or fastened to the table, is “temporarily disposed” on the table.

As used herein, the term “correspond” indicates that two structural components are sized and shaped to be similar to each other and is coupled with a minimum amount of friction. Thus, an opening which “corresponds” to a member is sized slightly larger than the member so that the member may pass through the opening with a minimum amount of friction. This definition is modified if the two components are to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines, two, or more, “corresponding” surfaces, shapes, or lines have generally the same size, shape, and contours.

As used herein, a “path of travel” or “path,” when used in association with an element that moves, includes the space an element moves through when in motion. As such, any element that moves inherently has a “path of travel” or “path.”

As used herein, the statement that two or more parts or components “engage” one another shall mean that the elements exert a force or bias against one another either directly or through one or more intermediate elements or components. Further, as used herein with regard to moving parts, a moving part may “engage” another element during the motion from one position to another and/or may “engage” another element once in the described position. Thus, it is understood that the statements, “when element A moves to element A first position, element A engages element B,” and “when element A is in element A first position, element A engages element B” are equivalent statements and mean that element A either engages element B while moving to element A first position and/or element A either engages element B while in element A first position.

As used herein, the term “operatively engage” means “engage and move.” That is, “operatively engage” when used in relation to a first component that is structured to move a movable or rotatable second component means that the first component applies a force sufficient to cause the second component to move. For example, a screwdriver is placed into contact with a screw. When no force is applied to the screwdriver, the screwdriver is merely “coupled” to the screw. If an axial force is applied to the screwdriver, the screwdriver is pressed against the screw and “engages” the screw. However, when a rotational force is applied to the screwdriver, the screwdriver “operatively engages” the screw and causes the screw to rotate. Further, with electronic components, “operatively engage” means that one component controls another component by a control signal or current.

As used herein, the term “number” shall mean one or an integer greater than one (e.g., a plurality).

As used herein, in the phrase “[x] moves between its first position and second position,” or, “[y] is structured to move [x] between its first position and second position,” “[x]” is the name of an element or assembly. Further, when [x] is an element or assembly that moves between a number of positions, the pronoun “its” means “[x],” i.e., the named element or assembly that precedes the pronoun “its.”

As used herein, a “radial side/surface” for a circular or cylindrical body is a side/surface that extends about, or encircles, the center thereof or a height line passing through the center thereof. As used herein, an “axial side/surface” for a circular or cylindrical body is a side that extends in a plane extending generally perpendicular to a height line passing through the center. That is, generally, for a cylindrical soup can, the “radial side/surface” is the generally circular sidewall and the “axial side(s)/surface(s)” are the top and bottom of the soup can.

As used herein, a “diagnostic” test includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject having a cancer or the likelihood that such a subject will respond favorably to a compound (e.g., a pharmaceutical, e.g., a drug) or other treatment.

As used herein, the term “condition” refers generally to a disease, malady, injury, event, or change in health status.

As used herein, the term “treating” or “treatment” with respect to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, “treatment” comprises exposing a patient or a portion thereof (e.g., a tissue, organ, body part, or other localize region of a patient body) to radiation (e.g., electromagnetic radiation, ionizing radiation).

As used herein, the term “beam” refers to a stream of radiation (e.g., electromagnetic wave and/or or particle radiation). In some embodiments, the beam is produced by a source and is restricted to a small-solid angle. In some embodiments, the beam is collimated. In some embodiments, the beam is generally unidirectional. In some embodiments, the beam is divergent.

As used herein, the term “patient” or “subject” refers to a mammalian animal that is identified and/or selected for imaging and/or treatment with radiation. Accordingly, in some embodiments, a patient or subject is contacted with a beam of radiation, e.g., a primary beam produced by a radiation source. In some embodiments, the patient or subject is a human. In some embodiments, the patient or subject is a veterinary or farm animal, a domestic animal or pet, or animal used for clinical research. In some embodiments, the subject or patient has cancer and/or the subject or patient has either been recognized as having or at risk of having cancer.

As used herein, the term “treatment volume” or “imaging volume” refers to the volume (e.g., tissue) of a patient that is selected for imaging and/or treatment with radiation. For example, in some embodiments, the “treatment volume” or “imaging volume” comprises a tumor in a cancer patient. As used herein, the term “healthy tissue” refers to the volume (e.g., tissue) of a patient that is not and/or does not comprise the treatment volume. In some embodiments, the imaging volume is larger than the treatment volume and comprises the treatment volume.

As used herein, the term “radiation source” or “source” refers to an apparatus that produces radiation (e.g., ionizing radiation) in the form of photons (e.g., described as particles or waves). In some embodiments, a radiation source is a linear accelerator (“linac”) that produces x-rays or electrons to treat a cancer patient by contacting a tumor with the x-ray or electron beam. In some embodiments, the source produces particles (e.g., photons, electrons, neutrons, hadrons, ions (e.g., protons, carbon ions, other heavy ions)). In some embodiments, the source produces electromagnetic waves (e.g., x-rays and gamma rays having a wavelength in the range of approximately 1 pm to approximately 1 nm). While it is understood that radiation can be described as having both wave-like and particle-like aspects, it is sometimes convenient to refer to radiation in terms of waves and sometimes convenient to refer to radiation in terms of particles. Accordingly, both descriptions are used throughout without limiting the technology and with an understanding that the laws of quantum mechanics provide that every particle or quantum entity is described as either a particle or a wave.

As used herein, the term “static source” refers to a source that does not revolve around a patient during use of the source for imaging or therapy. In particular, a “static source” remains fixed with respect to an axis passing through the patient while the patient is being imaged or treated. While the patient may rotate around said axis to produce relative motion between the static source and rotating patient that is equivalent to the relative motion of a source revolving around a static patient, a static source does not move with reference to a third object, frame of reference (e.g., a treatment room in which a patient is positioned), or patient axis of rotation during imaging or treatment, while the patient is rotated with respect to said third object, said frame of reference (e.g., said treatment room in which said patient is positioned), or patient axis of rotation through the patient during imaging or treatment. Thus, a static source is installed on a mobile platform and thus the static source may move with respect to the Earth and fixtures on the Earth as the mobile platform moves to transport the static source. Thus, the term “static source” may refer to a mobile “static source” provided that the mobile “static source” does not revolve around an axis of rotation through the patient during imaging or treatment of the patient. Further, the static source may translate and/or revolve around the patient to position the static source prior to imaging or treatment of the patient or after imaging or treatment of the patient. Thus, the term “static source” may refer to a source that translates or revolves around the patient in non-imaging and non-treatment use, e.g., to position the source relative to the patient when the patient is not being imaged and/or treated. In some embodiments, the “static source” is a photon source and thus is referred to as a “static photon source”.

### Description

Conventional arm rests for horizontal scanning and for vertical scanning are complicated and considerable time is required both to set up the arm rests prior to imaging or treating a patient and to position the patient on the patient support and arm rests. One problem with conventional arm rests that contributes to their complexity is the large number of index points and movements required for set up (see, e.g., Int'l Pat. Pub. No. WO2018120604). Accordingly, patient throughput is reduced and fewer patients are able to obtain the diagnosis and treatment they require. Lower patient throughput also results in an unfavorable cost-income balance for facilities that manage conventional CT scanning and X-ray treatment systems because the fixed costs of managing the CT scanning and X-ray treatment system are not offset by sufficient payments for its use.

The arm support assembly disclosed herein has fewer parts, fewer index points, and is simpler to move and set up than conventional technologies. The configurable arm support disclosed herein comprises a pair of brackets (or clamps) that interface with (attach to) and move substantially vertically along the lateral edges of a patient positioning system backrest to adjust the vertical height of the arm support (e.g., above the floor). An arm support arch comprises a rod with two linear portions (e.g., “legs”), each of which interfaces with one of the pair of brackets. The linear portions slide into and out from the brackets to adjust the position of the arm support with respect to the plane of the backrest. The arm support provides the supporting structure to support the arms (e.g., triceps) of the patient. Position of the arm support with respect to the plane of the backrest is indexed using a linearly arranged series of complementary protrusions (“bumps” or “pegs”) on the linear portions (e.g., arch legs) that fit into a hole on each bracket (a detent arrangement); vertical position of the arm rest is indexed using a linearly arranged series of complementary protrusions on the backrest and a hole on each bracket (a detent arrangement). Thus, the illustrated embodiment has only two index points. Protrusions and holes may be swapped among interfacing components to provide an alternative arrangement. Advantageously, the arm support assembly has no more than two index points and/or has gravity at least partially hold the arm support rod in place.

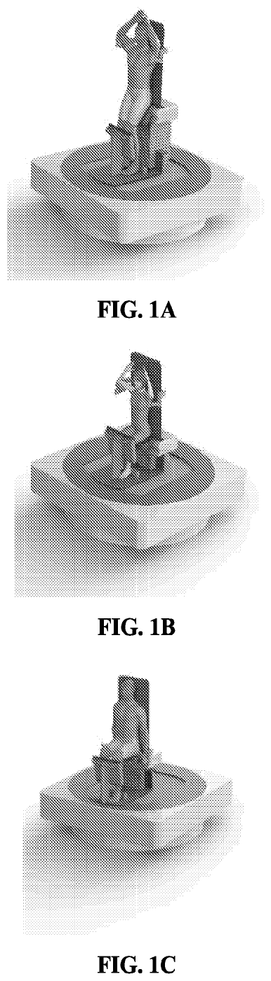

With reference to FIGS. 1A, 1B, and 1C, arm support needs vary for different upright treatment patent positions. FIG. 1A illustrates a chair supporting a patient in a perched position for treatment in the pelvis or lower abdomen, for example. FIG. 1B illustrates a chair supporting a patient for treatment in the thorax or upper abdomen, for example. FIG. 1C illustrates a chair supporting a patient for treatment in the head and neck. Arm support may vary for each treatment position. For treatment regions between the abdomen down to the genital area, a single support for the patient arms disclosed herein is set up quickly whilst providing the support required without overcomplicating the setup with multiple indexing points.

Controlling arm placement is recommended for all treatments to reduce movement to improve reproducibility of anatomical position. The arm support detailed herein is comfortable for the patient for the duration of the session whether imaging or treatment (approximately 15-20 minutes). The arm support detailed herein is positioned safely and securely by a clinician in an efficient manner to reduce the time taken for each session and increase throughput of patients. The arm support detailed herein is compatible with various 3^{rd} party equipment, for example, the backrest.

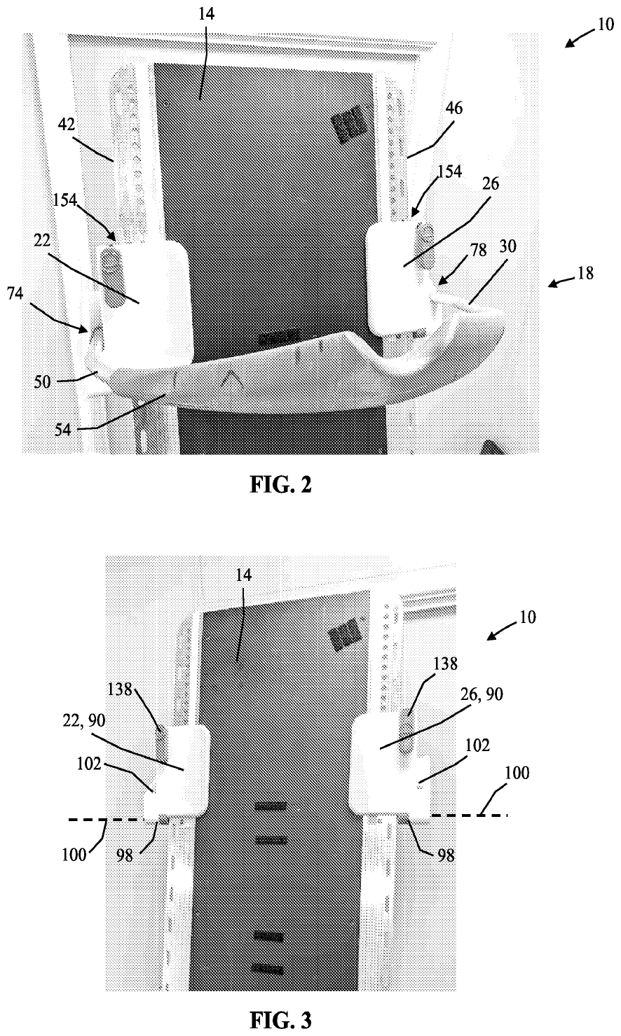

With reference to FIG. 2, an adjustable support assembly 10 configured to support a patient is illustrated. The adjustable support assembly 10 includes a generally vertical backrest 14 (e.g., a vertical table) and an arm support assembly 18 adjustably coupled to the backrest 14. In the illustrated embodiment, the arm support assembly 18 includes a first bracket assembly 22, a second bracket assembly 26, and an arm support 30.

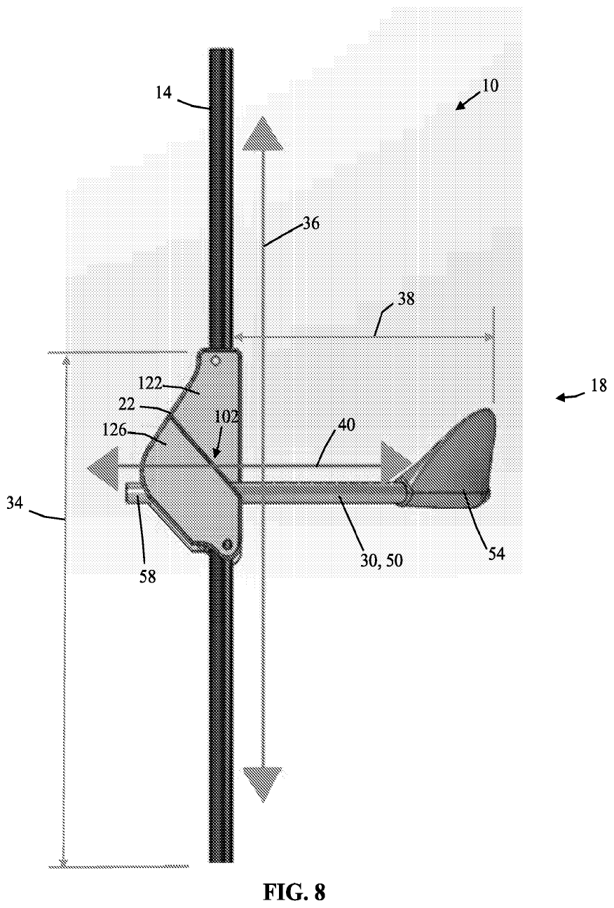

With reference to FIG. 8, as detailed further herein, the adjustable support assembly 10 includes two adjustable dimensions that are adjusted and tailored for each patient. In other words, the arm support assembly 18 is adjustable with respect to the backrest 18 in two dimensions (e.g., two indexes). A first adjustable dimension 34 for the patient is defined between the bracket assemblies 22, 26 and the backrest 14 (e.g., a bottom edge of the backrest) (e.g., an adjustable height). The first adjustable dimension 34 is adjustable along an adjustment axis 36. A second adjustable dimension 38 for the patient is defined between the arm support 30 and the backrest 14 (e.g., an adjustable width or spacing). The second adjustment dimension 38 is adjustable along an adjustment axis 40. In the illustrated embodiment, the adjustment axis 36 is perpendicular to the adjustment axis 40. Advantageously, the first adjustable dimension 34 and the second adjustable dimension 38 are the only adjustable dimensions for locating the arm support 30 with respect to the patient. In other words, the arm support assembly 18 is quickly and easily adjusted to meet the needs of different patients, thereby improving efficiency and repeatability.

With reference to FIG. 3, the first bracket assembly 22 is adjustably coupled to the backrest 14, and the second bracket assembly 26 is adjustably coupled to the backrest 14. In the illustrated embodiment, the first bracket assembly 22 is coupled to a first edge 42 of the backrest 14 and the second bracket assembly 26 is coupled to a second edge 46, opposite the first edge 42. In some embodiments, the backrest 14 is a conventionally horizontal table (e.g., a CIVCO table) that has been re-oriented to be generally vertical. In some embodiments, the arm support assembly 18 works with CIVCO indexing (a series of numbers and letters indicating where parts are positioned). In some embodiments, the backrest 14 is a curved back rest.

Bracket assemblies 22, 26 can extend out from the back of the back rest or from the front of the backrest 14. Advantageously, the bracket assemblies 22, 26 extend out from the front of the backrest 14 and provide extra structural support when the arm support 30 is extended far away from the body and provides additional room inside the scanner ring to accommodate the arm support. In other words, when the bracket assemblies extend from the front, the backrest can be located closer to the scanner ring, providing more room for the arm support.

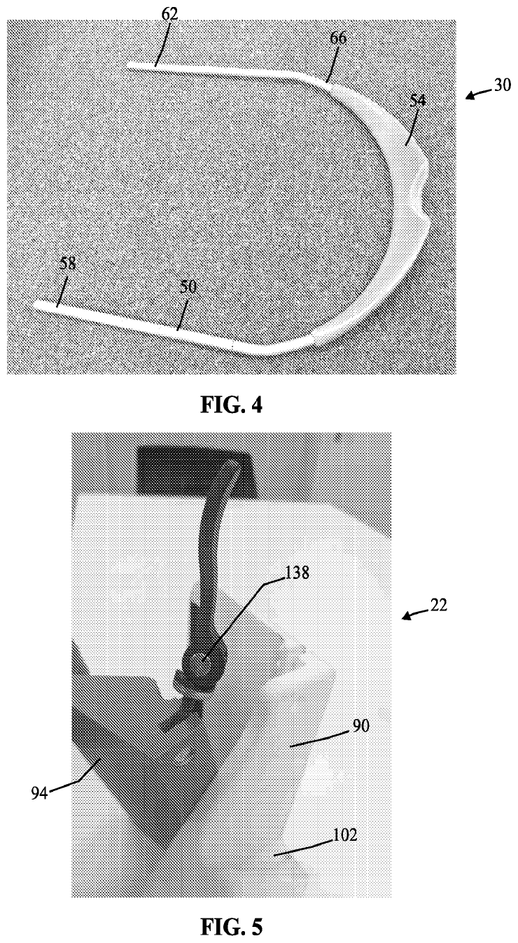

With reference to FIG. 4, the arm support 30 includes a rod 50 and a tray 54 coupled to the rod 50. In the illustrated embodiment, the rod 50 has a first linear portion 58, a second linear portion 62, and a connecting portion 66 positioned between the first linear portion 58 and the second linear portion 62. In other words, the connecting portion 62 extends between the first linear portion 58 and the second linear portion 62. In the illustrated embodiment, the connecting portion 66 is arcuate (e.g., an arch). In other embodiments, the connecting portion is linear. In some embodiments, the rod 50 is made of carbon fiber to minimize attenuation of the beam and minimize scatter danger.

With reference to FIG. 4, the tray 54 is coupled to the connecting portion 66 of the rod 50. The tray 54 provides a comfortable surface upon which the patient can rest their arm (e.g., triceps). In some embodiments, the tray 54 is formed as two separate pieces attached to the rod 50. In some embodiments, no tray is used.

As detailed herein, the arm support 30 is adjustably coupled to the first bracket assembly 22 and adjustably coupled to the second bracket assembly 26. In the illustrated embodiment, the first linear portion 58 of the rod 50 is at least partially received within the first bracket assembly 22, and the second linear portion 62 of the rod 50 is at least partially received within the second bracket assembly 26.

With reference to FIG. 8, the second adjustable dimension 38 between the arm support 30 and the bracket assemblies 22, 26 and backrest 14 is adjustable (e.g., according to a first index) along the adjustment axis 40. As illustrated, the second adjustable dimension 38 is defined between the backrest 14 and the connecting portion 66 of the rod 50.

The first bracket assembly 22 and the rod 50 form a first detent arrangement 74, and the second bracket assembly 26 and the rod 50 form a second detent arrangement 78. In the illustrated embodiment, the first detent arrangement 74 includes a protrusion 82 (e.g., detent) on the first bracket assembly 22 and at least one aperture 86 (FIG. 7) on the rod 50.

In some embodiments, the apertures 86 are spaced approximately 1 cm apart. The second detent arrangement 78 includes a similar arrangement. In other embodiments, the location of the protrusion and apertures are reversed.

In the illustrated embodiment, the first bracket assembly 22 and the second bracket assembly 26 are substantially similar. Details of the first bracket assembly 22 and the second bracket assembly 26 are provided herein, with similar details being applicable to the other but not necessarily repeated herein. As such, the following description refers to “a bracket assembly 22” generically which represents either the first bracket assembly 22 and/or the second bracket assembly 26.

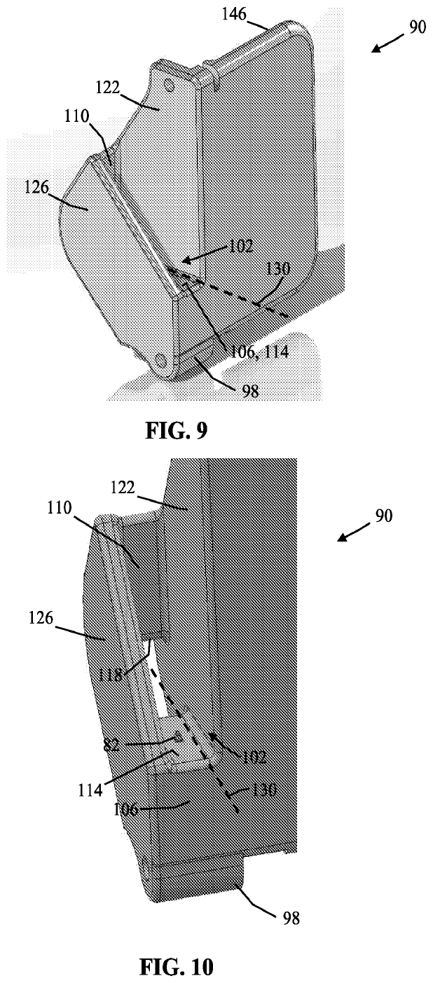

With reference to FIGS. 9-12, the bracket assembly 22 includes a first panel 90 and a second panel 94 coupled to the first panel 90 at a pivot 98.

With reference to FIG. 7, a support channel 102 is configured to receive at least a portion of the arm support 30. In the illustrated embodiment, the first panel 90 includes a first protrusion 106 (e.g., a lower support leg) and a second protrusion 110 (e.g., an upper support leg), and the support channel 102 is at least partially defined by the first protrusion 106 and the second protrusion 110. In the illustrated embodiment, the protrusions 106, 110 are spaced apart in at least two orthogonal directions and extend in generally parallel directions. The detent 82 (e.g., a protrusion) is positioned within the support channel 102.

With continued reference to FIG. 7, the first protrusion 106 of the support channel 102 has a first surface 114 configured to engage the arm support 30. The second protrusion 110 of the support channel 102 has a second surface 118 configured to engage the arm support 30. The first surface 114 is spaced from and facing towards the second surface 118. In the illustrated embodiment, the first surface 114 faces generally upwards, and the second surface 118 faces generally downwards. In the illustrated embodiment, the detent 82 is formed on the first surface 114 of the first protrusion 106.

With reference to FIGS. 9, 10, and 11, the first panel 90 includes a first side portion 122 extending between the first protrusion 106 and the second protrusion 110. The first panel 90 also includes a second side portion 126 extending between the first protrusion 106 and the second protrusion 110. In the illustrated embodiment, the first side portion 122 is spaced from and parallel to the second side portion 126. In other words, the protrusions 106, 110 extend between the parallel side portions 122, 126. The support channel 102 is at least partially defined between the first side portion 122, the second side portion 126, the first protrusion 106, and the second protrusion 110.

With reference to FIG. 7, the linear portion 58, 62 of the arm support 30 is at least partially received within the support channel 102, and the linear portion 58 engages both the first surface 114 and the second surface 118. The support channel 102 defines a support axis 130 that extends along the support channel 102, and the linear portion 58, 62 is aligned with the support axis 130 in FIG. 7.

In some embodiments, the arm support 30 is heavier in the front (e.g., near the connecting portion 66). As such, gravity acting on arm support 30 and/or acting on both arm support 30 and patient arms/body locks the arm support 30 into place in the support channels 102 of the bracket assemblies 22, 26.

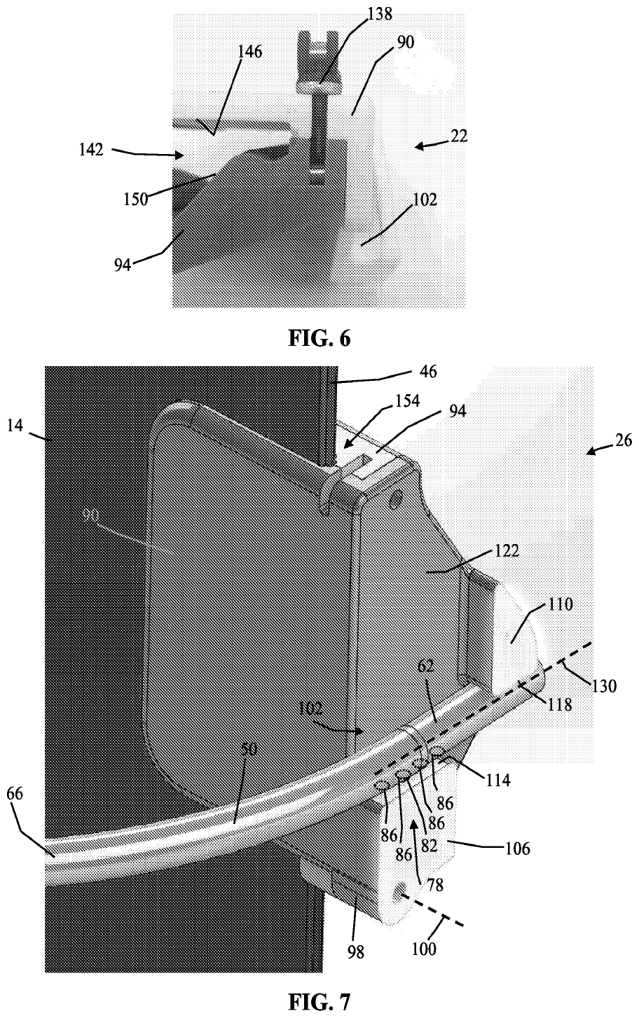

As detailed herein, the bracket assembly 22 is movable between a first configuration (e.g., an open configuration, FIG. 5) where the first panel 90 is movable relative to the second panel 94 about the pivot 98; and a second configuration (e.g., a locked configuration, FIG. 6) where the first panel 90 is fixed relative to the second panel 94. In the illustrated embodiment, the pivot 98 defines a pivot axis 100 the panels 90, 94 pivot about the pivot axis 100. In the illustrated embodiment, the support axis 130 of the support channel 102 is perpendicular to the pivot axis 100.

In the first configuration, the first panel 90 is pivoted further away from the second panel 94 when compared to the second configuration. In other words, a distance between the first panel 90 and the second panel 94 is larger in the first configuration than in the second configuration. In the illustrated embodiment, the top edge of the panels 90, 94 are positioned closer together when in the second configuration (FIG. 6) than when in the first configuration (FIG. 5).

A fastener 138 is provided to selectively place the bracket assembly 22 in the second configuration. In the illustrated embodiment, the fastener 138 is a cam lever handle that is manually actuated by a user. In the illustrated embodiment, the cam lever handle is fixed to the second panel 94 and is at least received through the first panel 90 in the second configuration.

With reference to FIGS. 6 and 7, a clamping channel 142 is at least partially defined between the first panel 90 and the second panel 94. In the illustrated embodiment, the clamping channel 142 is at least partially defined by a planar surface 146 on the first panel 90 and an arcuate surface 150 on the second panel 94. The planar surface 146 is positioned opposite from and facing towards the arcuate surface 150. In other words, the space formed between the planar surface 146 and the arcuate surface 150 forms the clamping channel 142 that receives and is secured to the backrest 14.

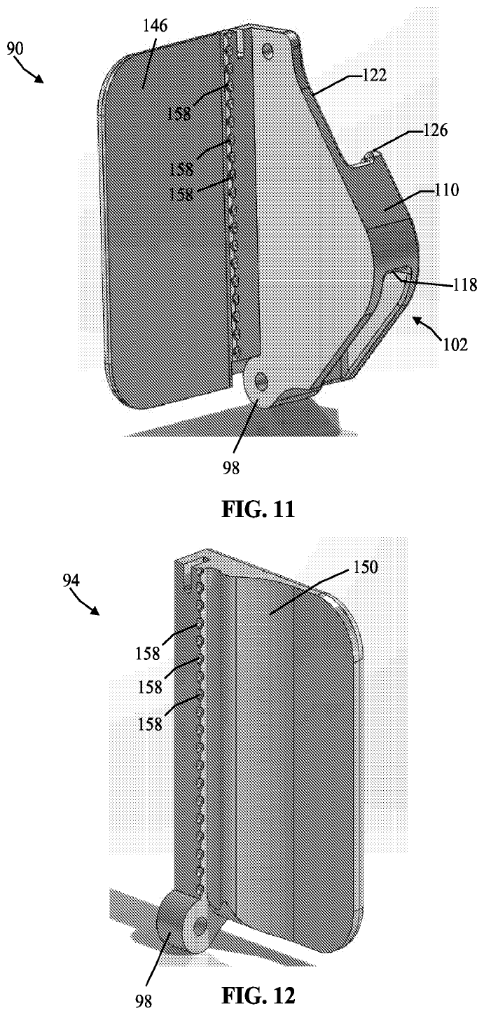

The bracket assembly 22 and the backrest 14 form a detent arrangement 154. In the illustrated embodiment, the detent arrangement 154 includes protrusions (e.g. bumps, pegs, etc.) formed on the edges 42, 46 of the backrest 14 and corresponding recesses 158 (e.g., indents) form on the bracket assembly 22. With reference to FIGS. 11 and 12, the recesses 158 are formed in the clamping channel 142. In the illustrated embodiment, the recesses 158 are at least partially formed by the first panel 90 and the second panel 94 when the bracket assembly is in the second configuration. In other words, the recesses 158 are partially formed in the first panel 90 and partially formed in the second panel 94. In some embodiments, the spacing of the protrusions on the backrest 14 is approximately 7 cm apart.

In some embodiment, the linear portions 58, 62 of the rod 50 are different lengths for different patients. In some embodiments, an extender piece can screw into the linear portion to make them longer.

Some embodiments include an optical system to confirm the placement of bracket assemblies 22, 26 and the arm support 30 are at the correct location for a given patient. In some embodiments, an alert (e.g., visual, audio, or haptic) is provided if the arm support 30 is not set up correctly. In some embodiments, a projector projects guide marks where brackets and arm support should be set.

In some embodiments, the arm support can be “broken” into two halves for independent movement of each half (e.g., for breast imaging or treatment). Halves may swivel in plane parallel to floor (apart/out and away from patient) or may swivel down or up (in plant parallel to back rest).

In some embodiments, the arm support provides an alert (haptic, sound) when patient breathing pattern is straying from prescribed pattern needed for imaging or treatment. In some embodiments, the arm support indicates a prescribed rhythm. The alert can vary in intensity depending on how far from proper pattern patient breathing is.

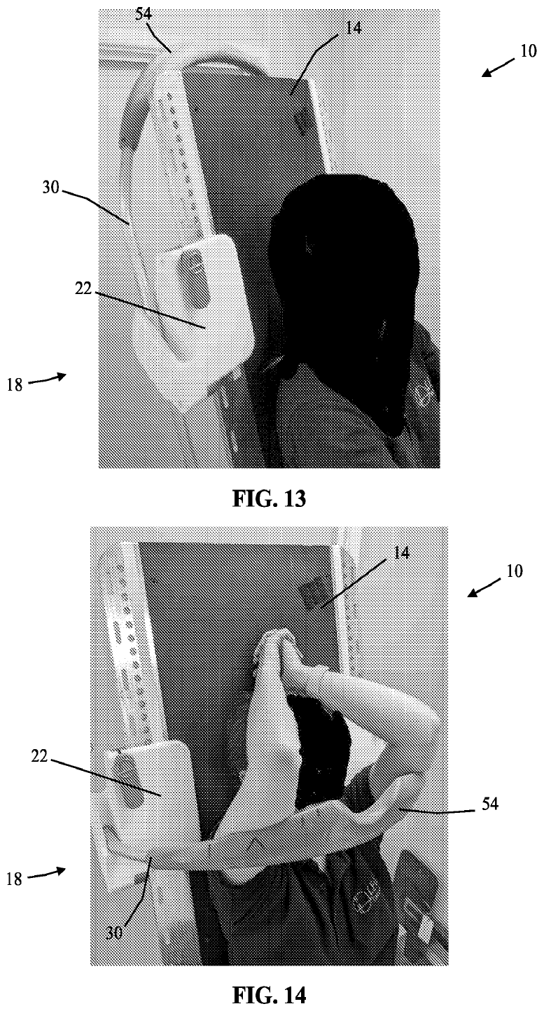

The present disclosure provides a method of adjusting a patient support to accommodate a patient that is an improvement from conventional methods. The method includes adjusting a first height of a first bracket assembly with respect to a backrest, and adjusting a second height of a second bracket assembly with respect to the backrest. The method also includes inserting an arm support at least partially in the first bracket assembly and at least partially in the second bracket assembly. The method then includes adjusting a spacing of the arm support with respect to the backrest. In other words, the arm support is moved with respect to the bracket assemblies and backrest. In some embodiments, adjusting the spacing of the arm support with the backrest is perpendicular to adjusting the first heigh of the first bracket assembly with respect to the backrest. In some embodiments, adjusting the heights of the bracket assemblies is vertical, and adjusting the spacing of the arm support from the backrest is horizontal (see, for example, FIG. 8). In some embodiment, the method further includes moving the arm support between an in-use position (e.g., FIG. 14) and a storage position (e.g., FIG. 13). In the in-use position, the linear portions 58, 62 of the rod 50 are aligned with the support axis 130 of the corresponding support channel 102. In some embodiment, the storage position has the arm support completely separated from (e.g., removed from) the bracket assemblies.

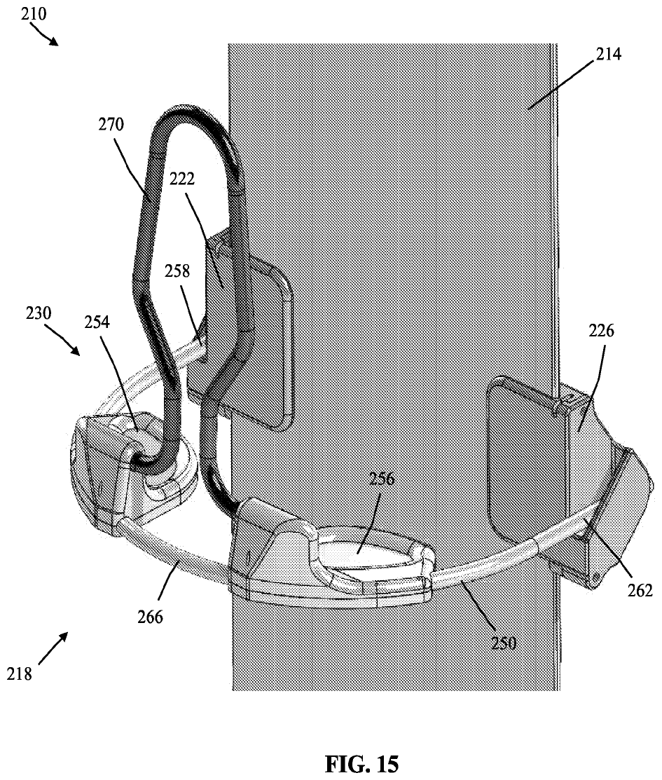

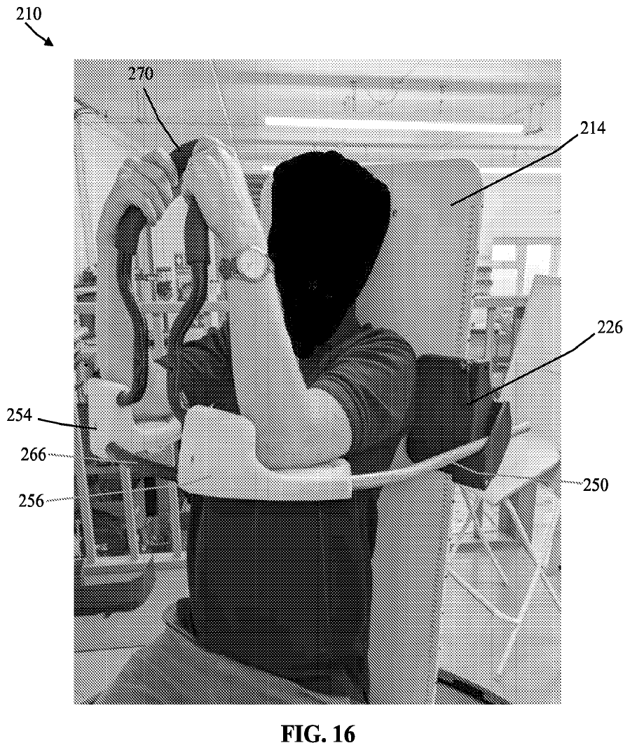

With reference to FIGS. 15 and 16, an adjustable support assembly 210 includes a generally vertical backrest 214 (e.g., a vertical table) and an arm support assembly 218 adjustably coupled to the backrest 214. In the illustrated embodiment, the arm support assembly 218 includes a first bracket assembly 222, a second bracket assembly 226, and an arm support 230. The arm support 230 includes a rod 250, a first tray 254 coupled to the rod 250, and a second tray 256 coupled to the rod 250.

In the illustrated embodiment, the rod 250 has a first linear portion 258, a second linear portion 262, and a connecting portion 266 positioned between the first linear portion 258 and the second linear portion 262. In other words, the connecting portion 262 extends between the first linear portion 258 and the second linear portion 262. In the illustrated embodiment, the connecting portion 266 is arcuate (e.g., an arch). The trays 254, 256 are coupled to the connecting portion 266 of the rod 250. In the illustrated embodiment, the first ray 254 is spaced apart from the second tray 256. In some embodiments, each of the trays 254, 256 includes a cushion (e.g. a gel pad) to improve patient comfort.

In the illustrated embodiment, the arm support 230 further includes a handle grip 270 extending between the first tray 254 and the second tray 256. In some embodiments, the handle grip 270 is movable with respect to the first tray 254 and the second tray 256. For example, the handle grip 270 may be foldable for transport and/or storage. In use, the handle grip 270 may extend generally vertical and/or generally parallel with the backrest 214.

Although the disclosure herein refers to certain illustrated embodiments, it is to be understood that these embodiments are presented by way of example and not by way of limitation.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

What is claimed is:

1. A bracket assembly for an arm support, the bracket assembly comprising:
a first panel;
a second panel coupled to the first panel at a pivot,
a support channel configured to receive at least a portion of the arm support; and
a clamping channel at least partially defined between the first panel and the second panel,
wherein the bracket assembly is movable between a first configuration where the first panel is movable relative to the second panel about the pivot and a second configuration where the first panel is fixed relative to the second panel.

2. The bracket assembly of claim 1, further including a fastener to selectively place the bracket assembly in the second configuration.

3. The bracket assembly of claim 1, wherein the first panel includes a first protrusion and a second protrusion and the support channel is at least partially defined by the first protrusion and the second protrusion.

4. The bracket assembly of claim 3, wherein the first protrusion has a first surface configured to engage the arm support, and the second protrusion has a second surface configured to engage the arm support, wherein the first surface is spaced from and facing towards the second surface.

5. The bracket assembly of claim 4, further including a detent formed on the first surface of the first protrusion.

6. The bracket assembly of claim 3, wherein the first panel includes a first side portion extending between the first protrusion and the second protrusion, and a second side portion extending between the first protrusion and the second protrusion, wherein the first side portion is spaced from and parallel to the second side portion.

7. The bracket assembly of claim 6, wherein the support channel is at least partially defined between the first side portion, the second side portion, the first protrusion, and the second protrusion.

8. The bracket assembly of claim 1, further including a detent positioned within the support channel.

9. The bracket assembly of claim 1, wherein in the first configuration the first panel is pivoted further away from the second panel than when in the second configuration.

10. The bracket assembly of claim 1, wherein the support channel defines a support axis and the pivot defines a pivot axis and wherein the support axis is perpendicular to the pivot axis.

11. The bracket assembly of claim 1, wherein the clamping channel is at least partially defined by a planar surface on the first panel and an arcuate surface of the second panel, wherein the planar surface is positioned facing towards the arcuate surface.

12. The bracket assembly of claim 1, further including a detent formed in the clamping channel.

13. The bracket assembly of claim 12, wherein the detent is at least partially formed by the first panel and the second panel when the bracket assembly is in the second configuration.

14. An arm support assembly comprising:
a first bracket assembly;
a second bracket assembly; and
an arm support with a rod having a first linear portion, a second linear portion, and a connecting portion positioned between the first linear portion and the second linear portion;
wherein the first linear portion is at least partially received in the first bracket assembly and the second linear portion is at least partially received in the second bracket assembly.

15. The arm support assembly of claim 14, wherein the arm support includes a tray coupled to the rod.

16. The arm support assembly of claim 15, wherein the tray is coupled to the connecting portion.

17. The arm support assembly of claim 16, wherein the tray is a first tray and the arm support includes a second tray coupled to the connecting portion, wherein the first tray is spaced apart from the second tray.

18. The arm support assembly of claim 17, further including a handle grip extending between the first tray and the second tray.

19. The arm support assembly of claim 18, wherein the handle grip is movable with respect to the first tray and the second tray.

20. The arm support assembly of claim 14, wherein a distance between the arm support and the first bracket assembly is adjustable.

21. The arm support assembly of claim 20, wherein the distance is between the first bracket assembly and the connecting portion.

22. The arm support assembly of claim 14, wherein the first bracket assembly and the rod form a first detent arrangement, and wherein the second bracket assembly and the rod form a second detent arrangement.

23. The arm support assembly of claim 22, wherein the first detent arrangement includes a protrusion on the first bracket assembly and an aperture on the rod.

24. The arm support assembly of claim 14, wherein the connecting portion is arcuate.

25. The arm support assembly of claim 14, wherein the arm support is movable with respect to the first bracket assembly and the second bracket assembly between an in-use position and a storage position.

26. An adjustable support assembly configured to support a patient, the adjustable support assembly comprising:
a backrest;
a first bracket assembly adjustably coupled to the backrest;
a second bracket assembly adjustably coupled to the backrest; and
an arm support adjustably coupled to the first bracket assembly and adjustably coupled to the second bracket assembly;
wherein a first adjustable dimension for the patient is defined between the first bracket assembly and the backrest; and
wherein a second adjustable dimension for the patient is defined between the arm support and the backrest.

27. The adjustable support assembly of claim 26, wherein the first adjustable dimension and the second adjustable dimension are the only adjustable dimensions for locating the arm support with respect to the patient.

28. The adjustable support assembly of claim 26, wherein the arm support includes a rod with a first portion and a second portion, wherein the first portion is at least partially received within the first bracket assembly and the second portion is at least partially received within the second bracket assembly.

29. The adjustable support assembly of claim 26, wherein the first bracket assembly is coupled to a first edge of the backrest and the second bracket assembly is coupled to a second edge of the backrest, the second edge opposite the first edge.

30. The adjustable support assembly of claim 26, wherein the backrest is vertical.

31. A method of adjusting a patient support to accommodate a patient, the method comprising:
adjusting a first height of a first bracket assembly with respect to a backrest;
adjusting a second height of a second bracket assembly with respect to the backrest;
inserting an arm support at least partially in the first bracket assembly and at least partially in the second bracket assembly; and
adjusting a spacing of the arm support with respect to the backrest.

32. The method of claim 31, wherein adjusting the spacing of the arm support with respect to the backrest is perpendicular to adjusting the first height of the first bracket assembly with respect to the backrest.

33. The method of claim 31, further including moving the arm support between an in-use position and a storage position.
